(19) United States
(12) Patent Application Publication
(10) Pub. No.: US 2025/0281372 A1
(43) Pub. Date: Sep. 11, 2025

(54) POWDERS HAVING IMPROVED DISPERSABILITY AND USE THEREOF IN COMPOSITIONS AND ARTICLES OF MANUFACTURE

(71) Applicant: Color Techniques, Inc., South Plainfield, NJ (US)

(72) Inventors: George Lambridis, Wayne, NJ (US); David Dick, Sparta, NJ (US); Carol Edridge, South Plainfield, NJ (US)

(21) Appl. No.: 18/859,060

(22) PCT Filed: Apr. 24, 2023
(86) PCT No.: PCT/US2023/066103

§ 371 Date: Oct. 22, 2024

(51) Int. Cl. A61K 8/11; A61K 8/19; A61K 8/26; A61K 8/365; A61K 8/55; A61K 8/893; A61Q 1/02

(52) U.S. Cl. CPC A61K 8/11; A61K 8/19; A61K 8/26; A61K 8/365; A61K 8/553; A61K 8/893; A61Q 1/02

(57) ABSTRACT

The present disclosure relates generally to powder materials having a surface treated with a unitary coating, compositions containing the coated powder, and methods of making powder having a surface treated with a unitary coating. The coated powders of the present invention are readily dispersible in non-polar liquid materials and have improved stability in cosmetic compositions.

### FIELD OF THE INVENTION

The present disclosure relates generally to powder materials having a surface treated with a unitary coating, compositions containing the coated powder, and methods of making powder having a surface treated with a unitary coating. The coated powders of the present invention are readily dispersible in non-polar liquid materials and have improved stability in cosmetic compositions.

### BACKGROUND

Various powders are conventionally used for making cosmetics, skincare products, toiletries, and other products marketed and distributed by the personal care industry. Powders dispersed in oil-based product forms may suffer from poor dispersibility and product stability, which can result in the formation of aggregates, agglomerates, and flocculation. These results can be due to the nature of the powder's physical properties, including particle size, surface activity, charge, polarity, and specific gravity, to name a few.

Inorganic pigments (e.g., ultramarines and manganese violet), metal oxides (e.g. iron oxides, titanium dioxide, zinc oxide, chromium oxide), organic pigments, and natural and synthetic minerals and fillers (e.g. mica, talc, silica, kaolin) can agglomerate easily due to several surface properties (including surface charge, surface polarity, etc.). In order to address this problem and to thereby improve the dispersibility and stability of powders in non-polar media, surface treatments with various treating agents have been proposed. Agents and methods for surface treating powders will vary depending on the aim of the treatment, and a particular treating agent may be selected in view of properties of the surface to be treated and its interaction with a dispersion medium.

Metal oxides of large or small particle size, even when coated like methicone and silane, are known to have poor dispersibility in esters, vegetable oil, mineral oil, and hydrocarbons. Poor dispersibility can adversely affect UV attenuation and transparency in sunscreens, and color strength in pigments, as well as skin feel and formulation stability.

U.S. Pat. No. 9,254,398 (Schlossman, et al.) discloses a series of dual-layer coated metal oxide powders. These coated powders are intended as ingredients for cosmetic compositions and articles of manufacture such as makeup, lipstick, nail enamel, eye shadow, and mascara. The first layer of this dual-layer coating is triethoxycaprylylsilane, and the second layer is polyhydroxystearic acid. While these powders demonstrate good dispersibility, their formulation requires a complicated process in which metal oxide powders are sequentially coated with two discrete layers of different coating materials. A simpler and more efficient solution is needed.

### SUMMARY OF THE INVENTION

According to one aspect of the present invention, there are provided powders having a unitary coating comprising a caprylic capric triglyceride (CCT) or other liquid oil carrier, a polyhydroxy fatty acid such as polyhydroxystearic acid, silicone, an aliphatic carboxylic acid such as isostearic acid, polyglyceryl-3 polyricinoleate, and lecithin. The surface treatment process utilizes a mixture of agents that bond and adsorb to the surfaces of the powder particles which allows coated particles to rapidly and efficiently disperse into non-polar media.

According to another aspect of the invention, there is provided a method for treating the surface of powder particles with a unitary coating composition comprising a CCT or other liquid oil carrier, a polyhydroxy fatty acid such as polyhydroxystearic acid, silicone, an aliphatic carboxylic acid such as isostearic acid, polyglyceryl-3 polyricinoleate, and lecithin. The surface treatment process of the present invention utilizes a mixture of agents that bond and adsorb to the surfaces of the powder particles allowing the coated particles to rapidly and efficiently disperse into the medium. While not bound by any particular theory, it is suggested that this rapid and efficient dispersion is accomplished with the aid of steric hindrance stabilization. Steric hindrance stabilization describes how a molecule's physical structure can affect its ability to react. When particles on a molecular level are “bulky,” that can hinder or even prevent other molecules from efficiently finding the desired site or positioning. By coating the particles with the coating treatment disclosed herein, the coated particles are prevented to re-agglomerating due to electrostatic forces, thus they are kept separated and stabilized.

According to another aspect of the invention, there are provided compositions and articles of manufacture containing powders treated with a unitary coating comprising a CCT or other liquid oil carrier, a polyhydroxy fatty acid such as polyhydroxystearic acid, silicone, an aliphatic carboxylic acid such as isostearic acid, polyglyceryl-3 polyricinoleate, and lecithin. Examples of such articles of manufacture include, but are not limited to, foundation, blush, eye shadow, eye liner, mascara, lipstick, lip liner, and the like.

### DETAILED DESCRIPTION

In one aspect of the present invention, there are provided powders having a unitary coating comprising a CCT or other liquid oil carrier, a polyhydroxy fatty acid, a silicone, an aliphatic carboxylic acid, polyglyceryl-3 polyricinoleate, and lecithin.

Powders suitable for use in the present invention include pigments. As used herein, the term “pigment” is a natural or synthetic material that has a certain color, shade, hue, chroma (saturation) or lightness. Pigments may be organic or inorganic in chemical nature. Pigments typically have a primary particle diameter not greater than about 3 μm. Pigments more typically are about 0.01-1.0 μm in diameter. Other pigments, such as pearl pigments typically have a larger size, for example 10, 20, 30, 40, or 50-100 μm.

Non-limiting examples of inorganic pigments include white titanium dioxide pigments (e.g., rutile, anatase, brookite, and ultrafine TiO₂), zinc oxides (e.g., ultrafine ZnO), zirconium oxides, zirconium dioxides, cerium oxides, iron oxides (including yellow, red, brown, green, and black iron oxides), ultramarines (such as ultramarine blue, ultramarine violet, ultramarine pink, etc.), pearl pigments (e.g., mica, titanated mica, bismuth oxychloride, etc.), manganese violet, Prussian blue, Persian blue, chromium oxides, chromium hydroxides, magnesium oxides, aluminum oxides, aluminum hydroxides, and carbon black. Non-limiting examples of organic pigments include β-carotene, carmine, chlorophyll, and the like.

Powders suitable for use in the present invention may also include sericite, talc, kaolin, synthetic mica, muscovite, phlogopite, epidolite, biotite, calcium carbonate, magnesium carbonate, calcium phosphate, barium sulfate, magnesium sulfate, silicic acid, silicic anhydride, magnesium silicate, aluminum silicate, aluminum magnesium silicate, calcium silicate, barium silicate, strontium silicate, silicon carbide, magnesium aluminate, magnesium metasilicate aluminate, chlorohydroxyaluminum, clays, bentonite, calcium sodium silicate, aluminum calcium sodium silicate, fumed silica, alumino-silicate minerals (zeolites), smectite, hydroxyapatite, ceramic powders (such as silicon nitride or boron nitride), nylon (e.g., nylon beads or nylon powder), and silica.

Powders suitable for use in the present invention may also include organic powders, non-limiting examples of which include a wool powder, a silk powder, a polyamide powder, a polyester powder, a polyethylene powder, a polypropylene powder, a polystyrene powder, a polyurethane powder, a benzoguanamine powder, a tetrafluoroethylene powder, a polymethyl methacrylate powder, a cellulose powder, a silk powder, a silicone powder, a silicone rubber powder, a styrene acrylic copolymer, a divinylbenzene-styrene copolymer, synthetic resin powders such as a vinyl resin, a urea resin, a phenol resin, a fluoro resin, a silicon resin, an acrylic resin, a melamine resin, an epoxy resin and a polycarbonate resin, a fine crystalline fibrous powder, a starch powder and starch derivatives such as aluminum starch octenylsuccinate, an acylated lysine powder, a long-chain alkyl phosphate metallic salt powder, or a metal soap powder.

Powders suitable for use in the present invention may also include CI 12085 (Red 36), CI 15850 (Red 6), CI 15850 (Red 7 Lake), CI 15985 (Yellow 6 Lake), CI 17200 (Red 33 Lake), CI 19140 (Yellow 5 Lake), CI 42090 (Blue 1 Lake), CI 45380 (Red 22 Lake), CI 45410 (Red 28 Lake), and CI 73360 (Red 30 Lake).

Powders suitable for use in the present invention may also include mixtures of two or more of any of the foregoing powder materials.

CCTs are produced by the esterification of glycerol (plant sugars) with mixtures of caprylic (C:8) and capric (C:10) fatty acids from coconut or palm kernel oils. The combination of fatty acids, and esterification, are responsible for its silky oil feel. The process used to achieve the skin benefits of the specific fatty acid esters also results in superior oxidative stability, low color, and odor, as it can then be further refined to remove residual fatty acids resulting in a pure ester with a silky oil feel. CCTs act as excellent emollients, dispersing agents and skin-replenishing ingredients, and are especially suited to sensitive and oily skin. CCTs are commonly used in skin care creams and lotions, make up, shampoos, and cleansers. Other suitable liquid oil carriers can include medium chain triglycerides (MCTs) that may include caproic (C:6) fatty acids. Suitable MCTs may include both straight chain and branched chain fatty acids. In the present invention, CCTs comprise from about 20 wt % to about 80 wt % of the coating composition.

Polyhydroxy fatty acids suitable for use in the coating composition of the present invention are polymers of hydroxy fatty acids contain from 14 to 24 carbon atoms. In one embodiment, the polyhydroxy fatty acid comprises polyhydroxystearic acid, an emulsifier and dispersant that is a polymerized from hydroxystearic acid and exists at room temperature as a viscous paste or as a gel. In such embodiment, polyhydroxystearic acid comprises from about 10 wt % to about 80 wt % of the coating composition.

Silicone, also called polysiloxane, comprises any of a diverse class of fluids, resins, or elastomers based on polymerized siloxanes, substances whose molecules consist of chains made of alternating silicon and oxygen atoms. In one embodiment, the type of silicone used in the coating composition is an alkoxyl alkyl silane. Non-limiting examples of suitable alkoxyl alkyl silanes include bis-stearoxydimethylsilane, stearoxytrimethylsilane, trimethoxycaprylylsilane, and triethoxycaprylylsilane. In one embodiment, the silicone is triethoxycaprylylsilane and comprises about 10 wt % to about 50 wt % of the coating composition.

Aliphatic carboxylic acids suitable for use in the coating composition of the present invention contain from 4 to 30 and preferably from 8 to 30 carbon atoms, and include hexanoic acid, heptanoic acid, octanoic acid, 2-ethylhexanoic acid, nonanoic acid, decanoic acid, undecanoic acid, dodecanoic acid, tridecanoic acid, tetradecanoic acid, pentadecanoic acid, hexadecanoic acid, hexyldecanoic acid, heptadecanoic acid, octadecanoic acid, isostearic acid, nonadecanoic acid, eicosanoic acid, isoatachidic acid, octyldodecanoic acid, heneicosanoic acid and docosanoic acid, and mixtures thereof. In one embodiment, the aliphatic carboxylic acid is isostearic acid and comprises from about 5 wt % to about 30 wt % of the coating composition.

Polyglyceryl-3 polyricinoleate is an ester of polyglycerin-3 and a polymer of ricinoleic acid and is used in as an emulsifier and dispersing agent. In one embodiment, polyglyceryl-3 polyricinoleate comprises from about 1 wt % to about 10 wt % of the coating composition.

Lecithins are phospholipids including phosphatidylcholine, phosphatidylethanolamine, phosphatidylinositol, phosphatidylserine, and phosphatidic acid, and are used as an emulsifier and lubricating agent. In one embodiment, the lecithin component comprises from about 1 wt % to about 10 wt % of the coating composition.

In one embodiment, the silicone component of the coating composition can be replaced with an ester in order to obtain a silicone-free final product. Suitable esters are preferably esters that are 100% naturally derived and that also possess excellent wetting properties. Non-limiting examples of such esters include coco-caprylate/caprate and trioctyldodecyl citrate.

The coating composition may be prepared by mixing the foregoing components in sequence in an appropriately sized vessel. The components may be mixed with a propeller mixer until a homogeneous and uniform dispersion is obtained. The powder substrate (e.g. metal oxide) is then loaded into an appropriate powder blender. Such blenders include, but not limited to a ribbon blender, PK V-blender, or twin-cone blender all equipped with spraying capabilities. The blending is initiated and, while mixing, the coating composition is evenly applied in a one-step process onto the powder and mixed until the surfaces of the powder substrate is coated uniformly. In one embodiment, the coating composition is sprayed via fine droplets in a one-step process onto the powder and mixed until the surfaces of the powder substrate are coated uniformly. The resulting coated powder is discharged and may be milled through a hammer mill or similar type of equipment for further particle development and even distribution. In one embodiment, the preferred weight percentage ranges for the resulting coated powder are as follows:

| | |
|---|---|
| Black Iron Oxide (CI 77499) | 90.00% to 98.00% |
| Caprylic/Capric Triglyceride | 0.50% to 5.00% |
| Polyhydroxystearic Acid | 0.50% to 5.00% |
| Triethoxycaprylylsilane | 0.50% to 4.00% |
| Polyglyceryl-3 Polyricinoleate | 0.10% to 2.00% |
| Isostearic Acid | 0.10% to 2.00% |
| Lecithin | 0.05% to 1.50% |

It is suggested that the silicone moiety of the solution anchors onto the particle's surface while the other ingredients of the mixture adsorb onto their surface. This results in a powder material that demonstrates hydrophobicity/lipophilicity and rapidly disperses itself with minimal energy in oils, esters, hydrocarbons and other non-polar media.

### Example: OptiSperse Hair Root Touch Up Hot Pour Stick

| Phase | Ingredient | % w/w |
|---|---|---|
| A | C12-15 Alkyl Benzoate | 21.40 |
| A | Octyldodecanol | 5.00 |
| A | Tribehenin | 7.00 |
| A | Polyethylene Octyldodecanol & Disteardimonium | 9.00 |
| | Hectorite & Propylene | |
| B | Carbonate | 4.50 |
| B | Caprylyl Glycol & Phenoxyethanol & Hexylene Glycol | 0.50 |
| B | Simethicone | 0.10 |
| C | Tapioca Starch & Polymethylsilsequioxane | 20.00 |
| C | Silica | 6.00 |
| D | Red Iron Oxide & Caprylic/Capric Triglyceride & | 2.00 |
| | Polyhydroxystearic Acid & Triethoxycaprylylsilane & | |
| | Isostearic Acid & Polyglyceryl-3 Polyricinoleate & | |
| | Lecithin | |
| D | Yellow Iron Oxide & Caprylic/Capric Triglyceride & | 1.50 |
| | Polyhydroxystearic Acid & Triethoxycaprylylsilane & | |
| | Isostearic Acid & Polyglyceryl-3 Polyricinoleate & | |
| | Lecithin | |
| D | Black Iron Oxide & Caprylic/Capric Triglyceride & | 11.00 |
| | Polyhydroxystearic Acid & Triethoxycaprylylsilane & | |
| | Isostearic Acid & Polyglyceryl-3 Polyricinoleate & | |
| | Lecithin | |
| D | Mica & Caprylic/Capric Triglyceride & | 12.00 |
| | Polyhydroxystearic Acid & Triethoxycaprylylsilane & | |
| | Isostearic Acid & Polyglyceryl-3 Polyricinoleate & | |
| | Lecithin | |
| | | 100.00 |

In this example, the ingredients identified as Phase A were heated to 85° C.-90° C. and mixed via a propeller mixer until melted and homogeneous. The next three ingredients-those identified as Phase B-were then added in sequence and mixed until homogeneous. The next two ingredients in Phase C were then added and mixed until homogeneous. The pigments identified as Phase D ingredients were then added to the coating composition, and mixed until homogeneous and uniform. The mixture was then poured at 80° C.-85° C. into appropriate packaging.

The compositions as described herein may be suitable for use in a variety of cosmetic compositions and cosmetics. As used herein, the term “cosmetic composition” means a composition that is intended to be applied onto a consumer's skin, particularly onto the facial skin or onto the body skin area or onto hair, so as to regulate the condition of the skin and/or to improve the appearance of the skin and/or hair. As used herein, the term “cosmetic” means a commercial product that contains a cosmetic composition.

Variations and modifications will occur to those of skill in the art after reviewing this disclosure. The disclosed features may be implemented, in any combination and sub-combinations (including multiple dependent combinations and sub-combinations), with one or more other features described herein. The various features described above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

As used throughout, ranges are used as a shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

Throughout this description, the use of the term “about” is intended to denote an approximation of the number, which includes the number modified by the term, and a reasonable deviation from that term, including standard measurement errors. Unless otherwise specified, all percentages and amounts expressed herein and elsewhere in the specification should be understood to refer to percentages by weight. The amounts provided are based on the active weight of the material. The recitation of a specific value herein is intended to denote that value, plus or minus a degree of variability to account for errors in measurements. For example, an amount of 10% or about 10% may include 9.5% or 10.5%, given the degree of error in measurement that will be appreciated and understood by those having ordinary skill in the art. Except in the operating examples, or where otherwise explicitly indicated, all numbers in this description indicating amounts of material or conditions of reaction, physical properties of materials and/or use are to be understood as modified by the word “about.”

As used herein % or wt % refers to percent by weight of an ingredient as compared to the total weight of the composition or component that is being discussed. All amounts are by weight of the final composition, unless otherwise specified.

We claim:

1. A composition, comprising:
a powder capable of rapidly dispersing in non-polar media, the powder comprising particles having a unitary coating wherein the unitary coating comprises a liquid oil carrier, a polyhydroxy fatty acid, silicone, an aliphatic carboxylic acid, polyglyceryl-3 polyricinoleate, and lecithin.

2. The composition of claim 1, wherein the powder comprises pigment.

3. The composition of claim 1, wherein the powder is selected from the group consisting of titanium dioxide, zinc oxide, zirconium oxide, zirconium dioxide, cerium oxide, iron oxide, ultramarine, pearl pigment, manganese violet, Prussian blue, Persian blue, chromium oxide, chromium hydroxide, magnesium oxide, aluminum oxide, aluminum hydroxide, carbon black, β-carotene, carmine, chlorophyll, and combinations thereof.

4. The composition of claim 1, wherein the powder is selected from the group consisting of sericite, talc, kaolin, synthetic mica, muscovite, phlogopite, epidolite, biotite, calcium carbonate, magnesium carbonate, calcium phosphate, barium sulfate, magnesium sulfate, silicic acid, silicic anhydride, magnesium silicate, aluminum silicate, aluminum magnesium silicate, calcium silicate, barium silicate, strontium silicate, silicon carbide, magnesium aluminate, magnesium metasilicate aluminate, chlorohydroxyaluminum, clay, bentonite, calcium sodium silicate, aluminum calcium sodium silicate, fumed silica, alumino-silicate, smectite, hydroxyapatite, ceramic powder, nylon, silica, and combinations thereof.

5. The composition of claim 1, wherein the powder is selected from the group consisting of wool powder, silk powder, polyamide powder, polyester powder, polyethylene powder, polypropylene powder, polystyrene powder, polyurethane powder, benzoguanamine powder, tetrafluoroethylene powder, polymethyl methacrylate powder, cellulose powder, silicone powder, silicone rubber powder, styrene acrylic copolymer, divinylbenzene-styrene copolymer, vinyl resin, urea resin, phenol resin, fluoro resin, silicon resin, acrylic resin, melamine resin, epoxy resin, polycarbonate resin, starch powder, aluminum starch octenylsuccinate, acylated lysine powder, a long-chain alkyl phosphate, metallic salt powder, metal soap powder, and combinations thereof.

6. The composition of claim 1, wherein the powder is selected from the group consisting of CI 12085 (Red 36), CI 15850 (Red 6), CI 15850 (Red 7 Lake), CI 15985 (Yellow 6 Lake), CI 17200 (Red 33 Lake), CI 19140 (Yellow 5 Lake), CI 42090 (Blue 1 Lake), CI 45380 (Red 22 Lake), CI 45410 (Red 28 Lake), CI 73360 (Red 30 Lake), and combinations thereof.

7. The composition of claim 1, wherein the liquid oil carrier is a caprylic capric triglyceride and wherein the caprylic capric triglyceride comprises from about 20 wt % to about 80 wt % of the coating.

8. The composition of claim 1, wherein the polyhydroxy fatty acid is a polymer of a hydroxy fatty acid containing from 14 to 24 carbon atoms.

9. The composition of claim 1, wherein the polyhydroxy fatty acid is polyhydroxystearic acid and wherein the polyhydroxystearic acid comprises from about 10 wt % to about 80 wt % of the coating.

10. The composition of claim 1, wherein the silicone is an alkoxyl alkyl silane.

11. The composition of claim 1, wherein the silicone is triethoxycaprylylsilane and wherein the triethoxycaprylylsilane comprises about 10 wt % to about 50 wt % of the coating.

12. The composition of claim 1, wherein the aliphatic carboxylic acid contains from 4 to 30 carbon atoms.

13. The composition of claim 1, wherein the aliphatic carboxylic acid is isostearic acid and wherein the isostearic acid comprises from about 5 wt % to about 30 wt % of the coating.

14. The composition of claim 1, wherein the polyglyceryl-3 polyricinoleate comprises from about 1 wt % to about 10 wt % of the coating.

15. The composition of claim 1, wherein the lecithin comprises from about 1 wt % to about 10 wt % of the coating.

16. A composition, comprising:
a powder capable of rapidly dispersing in non-polar media, the powder having a unitary coating wherein the unitary coating comprises a liquid oil carrier, a polyhydroxy fatty acid, an ester, an aliphatic carboxylic acid, polyglyceryl-3 polyricinoleate, and lecithin.

17. The composition of claim 16, wherein the ester is selected from the group consisting of coco-caprylate/caprate, trioctyldodecyl citrate, and combinations thereof.

18. A process for making coated pigment powder, comprising the steps of:
providing a pigment powder selected from the group consisting of titanium dioxides, zinc oxides, zirconium oxides, zirconium dioxides, cerium oxides, iron oxides, ultramarines, pearl pigments, manganese violet, Prussian blue, Persian blue, chromium oxides, chromium hydroxides, magnesium oxides, aluminum oxides, aluminum hydroxides, carbon black, β-carotene, carmine, chlorophyll, and combinations thereof;
applying a unitary coating comprising a liquid oil carrier, a polyhydroxy fatty acid, silicone, an aliphatic carboxylic acid, polyglyceryl-3 polyricinoleate, and lecithin to form coated pigment powder; and
optionally milling the coated pigment powder.

19. The method of claim 18, wherein the coating composition is sprayed via fine droplets in a one-step process onto the pigment powder and mixed until the surfaces of the pigment powder are coated uniformly.
